(19) United States
(12) Patent Application Publication
(10) Pub. No.: US 2023/0054094 A1
(43) Pub. Date: Feb. 23, 2023

(54) METHOD AND SYSTEM FOR PREPARING DIALYSIS FLUID FROM RAW WATER

(71) Applicants: BAXTER INTERNATIONAL INC., Deerfield, IL (US); BAXTER HEALTHCARE SA, Glattpark (Opfikon) (CH)

(72) Inventors: Tao HE, Shanghai (CN); Shuwei ZHAO, Shanghai (CN); Pengjia DOU, Shanghai (CN); Jianfeng SONG, Shanghai (CN); Markus STORR, Filderstadt (DE); Bernd KRAUSE, Rangendingen (DE)

(21) Appl. No.: 17/628,428

(22) PCT Filed: Jul. 20, 2020
(86) PCT No.: PCT/US2020/042783

§ 371 Date: Jan. 19, 2022

(30) Foreign Application Priority Data
Jul. 22, 2019 (CN) 2019106606997

(51) Int. Cl. A61M 1/14; B01D 61/00

(52) U.S. Cl. CPC A61M 1/14; B01D 61/002; A61M 1/1672

(57) ABSTRACT

The present disclosure relates to hollow fiber membrane filtration devices for the production of ready-to-use dialysis fluid by forward osmosis, and a cost-efficient and simple method and system for preparing ready-to-use dialysis fluid from raw water and liquid dialysis concentrate by forward osmosis.

### TECHNICAL FIELD

The present disclosure relates to hollow fiber membrane filtration devices for the production of ready-to-use dialysis fluid by forward osmosis, and a cost-efficient and simple method and system for preparing ready-to-use dialysis fluid from raw water and liquid dialysis concentrate by forward osmosis.

### DESCRIPTION OF THE RELATED ART

End-stage kidney disease (ESKD) is a leading cause of morbidity and mortality worldwide. Renal replacement therapies (RRT) such as peritoneal dialysis and hemodialysis are the main lifesaving therapies for patients with ESKD. In 2010, the number of patients was estimated to be between 4.902 and 9.701 million worldwide, and is expected to expand dramatically over the next few decades, including regions and patients with limited access to pure water and centers to provide such RRT. Hemodialysis is the most frequent used therapy for RRT patients, even though peritoneal dialysis also requires large amounts of dialysis fluids or the online generation of such fluids under consumption of pure water. Large amounts of ultrapure water are used for diluting the dialysis concentrate to prepare dialysate during a hemodialysis treatment. A patient needs about 120 to 200 liters of fluid over a 4-hour treatment session (Hoenich and Ronco, Blood Purif 2007; 25: 62-68), and discharge medical wastewater of the same quantity. The key operation involves the exchange of fluid and solutes between a patient's blood and a prepared fluid called dialysate. Dialysate fluid is typically generated by online mixing of dialysis concentrate (Part A) and a buffering agent (Part B) with a stream of ultrapure water, usually produced by a separate reverse osmosis (RO) system which is installed in the clinics or treatment centers. A typical dilution protocol is 1.0 part of Part A, 1.225 parts of Part B and 32.775 parts of ultrapure water. For any patient, a large quantity of pure water requires special caution for pyrogens, bacterial contaminations and storage etc. If the water consumption is greatly reduced, the development of portable dialysis is possible for both clinics, in-house or personal hemodialysis treatment, and patients could benefit from greater convenience, more freedom and a better life.

To install a small-scale RO machine for each patient in their house to produce pure water for dialysis is practically prohibited due to high instrument cost, high maintenance cost and potential contamination. However, some publications have described the use of RO for generating pure water from tap water for the preparation of dialysis fluid (U.S. Ser. No. 10/099,179 B2).

The expression “pure water” as used herein, refers to water which is purified and can be used in hemodialysis. Water for dialysis is required to contain <100 colony-forming unit/ml (CFU/ml) using sensitive microbiological methods and <0.5, preferably <0.25 endotoxin unit/ml (EU/ml) using the limulus amoebocyte lysate (lAl) assay. It is further defined by maximum allowable levels of toxic chemicals and dialysis fluid electrolytes and maximum allowable levels of trace elements as described in ISO13959:2014.

The inventors of the present application have therefore set out to explore the potential of forward osmosis (FO) as a potential low cost, low energy and low maintenance alternative. Driven by osmotic pressure gradient, FO is silent and ideal for medical instruments, especially in a home setting. Unlike pressure-driven RO process, FO membrane is less accessible to irreversible fouling and scaling (Chen et al., Desalination 366 (2015): 113-120), thus less backwashing or chemical cleaning agents are required. This approach has the potential to replace the current dialysate preparation practice by reducing the allocation of pure water from main clinic centers to patients. Smith et al. (J Mem Sci (2014): 469, 95-111) reported the use of a spiral-wound reverse osmosis polyamide membrane element for the dilution of dialysis concentrate by pre-treated tap water as a feed solution and has provided a number of theoretical consideration for such application. However, reports on using FO to dilute dialysis concentrate are otherwise very limited, even though the huge difference in the osmotic pressure between the highly saline dialysate concentrate and tap water is a solid justification for using osmotic dilution for this specific application. The use of FO has so far mainly been described for waste water management and desalination (U.S. Pat. No. 9,248,419 B2).

The use of FO for regeneration of spent dialysate has, in contrast, been described in several publications. For example, Talaat described the successful reclaiming of 38% of spent dialysis fluid water by forward osmosis through a cellulose triacetate (CTA) membrane (Talaat, Artificial Organs 33 (2009): 1133-1135). US 20170065762 A1 discloses the recycling of spent dialysate during renal replacement therapies for the preparation of fresh reconstitution fluid with FO membranes, wherein these FO membranes have nanoporous channels consisting, for example, of nanotubes or aquaporin water channels.

In forward osmosis treatment, the interior concentration polarization of the solute in the support layer has a major effect on the water permeation volume of the membrane. In forward osmosis treatment, a concentrated (draw) solution is situated on one side sandwiching the membrane, while a dilute (feed) solution is situated on the other side, and the difference in osmotic pressure between the two solutions is used as the driving force to cause migration of water from the feed side to the draw side. In order to increase the water permeation volume of the forward osmosis membrane during this time, it is important to maximally reduce the interior concentration polarization of the solute in the support layer reinforcing the thin membrane layer which exhibits the semi-permeable membrane performance, to increase the effective osmotic pressure difference of the thin membrane layer. Membranes for FO applications are known in the art. Conventional RO membranes are cellulose triacetate (CTA membranes), but also polysulfone based membranes have been described, wherein the porous layer is coated with a thin polyamide film. They are, therefore, referred to as thin film composite (TFC) FO membranes.

In WO 2012102678 A1, a forward osmosis membrane is described which can take the form of hollow fibers and which comprises a porous support layer and a thin film formed on the external side of the support layer. The membrane is used for desalination, waste water treatment as well as gas and food production.

WO 2017045983 A1 described a broad range of RO, microfiltration and FO membranes made from a broad spectrum of base materials and generally suggests their use in water treatment applications, desalination, plasmolysis, food processing and dialysis.

U.S. Pat. No. 9,193,611 B2 described TFC membranes comprising a substrate layer based on a sulfonated polymer and a polyamide film layer and suggests their use in FO, for example in waste water treatment, desalination, processing of pharmaceuticals and food and for potable water reuse devices.

EP 3181215 A1 discloses a forward osmosis membrane wherein a thin membrane layer exhibiting semi-permeable membrane performance is laminated on a polyketone support layer, wherein the thin membrane layer is made of cellulose acetate, polyamide, a polyvinyl alcohol/polypiperazineamide composite membrane, sulfonated polyethersulfone, polypiperazineamide or polyimide. The suggested membrane is used for power generation applications.

However, it remains a challenge to provide a cost-effective, stable and efficient FO membrane and device which can advantageously be used in a simple manner for production of pure water in medical applications, such as, for example, the generation of dialysis fluid from raw water.

In the present application, the inventors provide forward osmosis membranes which are cost-effective, simple to produce, and which with a surprisingly high efficiency can be used to prepare dialysis fluid using tap or raw water. Commercial CTA membranes were selected as a benchmark for analysis of the possibility of using osmotic dilution in hemodialysis and compared to thin film composite FO hollow fiber membranes based on commercially available hollow fiber hemodialysis membranes coated with a thin polyamide layer. To our best knowledge, this work is the first demonstration to apply FO membranes based on polyamide coated hemodialysis hollow fiber membranes for the preparation of dialysate with a scalable, controlled process, from raw water.

### SUMMARY

It is an object of the present invention to provide for a simple, cost efficient and easily accessible and producible hollow fiber membrane filtration device which can be used for the generation of ready-to-use dialysis fluid by forward osmosis, for example in situations or under conditions where no infrastructure for the generation of pure water by reverse osmosis is available. It was found by the inventors that it is possible to use known and commercially available hemodialyzers comprising state of the art high-flux polysulfone- or polyethersulfone-based hollow fiber membranes as forward osmosis membranes and devices after generally known interfacial polymerization of m-phenylenediamine and trimesoyl chloride on the lumen surface of the hollow fibers. The dialyzers, after said modification, can be efficiently used in a method for preparing ready-to-use dialysis fluid from raw water, such as, for example, tap water, by using said raw water as the feed solution and a dialysis concentrate as a draw solution. In said method, the raw water is fed into and passed through the lumen of the composite hollow fibers of the filtration device, while the dialysis concentrate is fed into and passed through the filtrate space of the filtration device, preferably in a countercurrent. According to another embodiment of the invention, the raw water is fed into and passed through the filtrate space of the filtration device, and the dialysis concentrate is fed into and passed through the lumen of the composite hollow fibers of the filtration device, preferably in a countercurrent.

This system further allows flexibility in terms of individually customized dialysate preparation, depending on when the dilution process is terminated.

The present application is specifically directed to a hollow fiber membrane filtration device (1) for the generation of ready-to-use dialysis fluid for use in hemodialysis by forward osmosis, comprising a plurality of hollow fibers (2) axially extending through a cylindrical housing (3) and being embedded and held, at their open ends, in a molding compound (4), thereby isolating said hollow fibers from a first fluid chamber (5) which is defined by the outer surface of said hollow fibers and the inner surface of said housing, wherein said first fluid chamber has an first fluid inlet (6) and a first fluid outlet (7), both provided on the housing, and further comprising a second fluid inlet (8) and a second fluid outlet (9) which are in communication with a second fluid chamber (10) which is defined by the interior of the hollow fibers, characterized in that the hollow fibers consist of a composite membrane comprising a hollow fiber support membrane which is comprised of 80-99% by weight of at least one hydrophobic polymer selected from the group consisting of polysulfone (PS), polyethersulfone (PES) and polyarylethersulfone (PAES), and 1-20% by weight of polyvinylpyrrolidone (PVP), and a polyamide layer on the lumen side of the hollow fiber support membrane, wherein the composite hollow fiber membrane provides for an average permeate flow rate of between 10.0 L/hm² to 20.0 L/hm².

The invention further relates to a method of preparing ready-to-use dialysis fluid from raw water by forward osmosis, comprising the steps of
a) providing a hollow fiber membrane filtration device as described above;
b) passing raw water through the second fluid chamber of the filter device, while recirculating a liquid dialysis concentrate through the first fluid chamber in a countercurrent, thereby creating a flux of pure water from the lumen side of the composite hollow fiber membrane into the dialysis concentrate;
c) terminating recirculation upon reaching the target concentration of the dialysis solution; and
d) providing the ready-to-use dialysis fluid for use in an extracorporeal hemodialysis circuit.

### BRIEF DESCRIPTION OF THE DRAWINGS

FIG. 1 is a schematic representation of the system for preparing ready-to-use dialysis fluid by FO, comprising a hollow fiber filtration device (1) wherein composite hollow fiber FO membranes (2) are distributed, a container (12) which provides for the initial dialysis concentrate (draw solution) and at the same time receives the diluted dialysis concentrate and the final ready-to-use dialysis solution, a source (19) for raw water (feed solution) and a container to receive the raw water after having passed the filtration device (1). The concentrate is pumped through a first chamber (5) of the filtration device in recirculation mode, whereas the raw water is fed into the lumen side of the hollow fibers which forms part of a second chamber (10) of the filtration device in countercurrent. An electrolyte detector (16) is used to determine when the desired dilution of the draw solution is reached.

FIG. 2 is a schematic representation of how the composite hollow fiber membranes are prepared. The process is generally well known as such and comprises contacting the interior (lumen) side of the hollow fiber support membranes with an aqueous MPD solution, thereby creating an aqueous MPD layer on the lumen of the fibers. In a next step, a TMC-hexane solution is passed through the lumen of the hollow fibers, and a polymerization reaction between MDP and TMC is started under suitable conditions, thereby forming a thin (5-500 nm range) polyamide layer on the lumen of the support membrane.

FIG. 3 shows SEM images of the cross section and surfaces of a commercial flat sheet CTA membrane and a composite HF-TFC membrane according to the invention. A. shows a CTA membrane cross section, B. shows the CTA membrane top surface. C. shows a composite HF-TFC membrane cross section, D. shows a composite HF-TFC membrane top surface. The cross-section of the CTA membrane sample was prepared by a surgery knife and thus appears rough.

FIG. 4 shows the zeta potential of a commercial flat sheet CTA membrane and of a composite HF-TFC hollow fiber membrane according to the invention depending on pH. All tests were conducted in triplicate and the error bars represent one standard deviation.

FIGS. 5A and B show the real rejections of commercial flat CTA (FIG. 5 A) and HF-TFC membranes according to the invention (FIG. 5 B) for typical ions of concern in the raw water as a function of reciprocal permeate flux. The system temperature was set to 25.0±0.5° C. Experimental conditions for the flat sheet CTA membrane: feed 10 L tap water, the operational pressure was 5.0-14.0 bar; for the composite hollow fiber membrane HF-TFC: feed 2 L tap water, the operational pressure was 2.0-5.0 bar.

FIG. 6 shows permeate flux (Jᵥ) as a function of dilution ratio and time (embedded graph) in the FO process. Feed solution: tap water (80 L); draw solution: dialysis concentrate (0.35 L); Temperature: 25.5±2.5° C. The permeate flux decreases with increasing dilution ratio of the draw solution. The permeate flux decreases very slightly over time in the case of HF-TFC membranes, which however show an overall higher permeate flux than CTA membranes.

FIG. 7 shows permeate flux (Jᵥ) as a function of time in the FO process. Feed solution: tap water (80 L); draw solution: dialysis concentrate (0.35 L); Temperature: 25.5±2.5° C. The permeate flux decreases with time.

### DETAILED DESCRIPTION

The present invention discloses devices and methods for forward osmosis (FO) as a potential low cost, low energy and low maintenance alternative for preparing ready-to-use dialysis fluid from tap water. Driven by osmotic pressure gradient, FO is silent and ideal for medical instruments. Unlike pressure-driven RO process, FO membranes are less prone to irreversible fouling and scaling, thus less backwashing or chemical cleaning agents is required. In consequence, forward osmosis also requires less maintenance. The disclosed approach is a viable alternative which could replace the current dialysate preparation practice by reducing the allocation of pure water from main clinic center to patients by local, point of care production of dialysis solutions from raw water and liquid dialysis concentrate.

The expression “forward osmosis (FO) membrane” or “forward osmosis (FO) hollow fiber membrane” as used herein refers to membranes which are adapted for use in forward osmosis, and specifically refers to asymmetric hollow fiber membranes comprising both phase-inversion and composite membrane components having a low support layer resistance of water transport, high water permeability, minimum reverse solute permeability and high selectivity for water molecules, enabling them to separate water molecules from other molecules, such as, for example, salts.

The expression “forward osmosis” as used herein refers to the transport of water molecules across a semipermeable membrane by osmotic pressure from a feed solution (FS) to a draw solution (DS), essentially in the absence of hydraulic pressure, wherein the draw solution has a high osmotic pressure and the feed solution has an osmotic pressure which is lower than the osmotic pressure of the draw solution.

In the context of the present invention, it is possible to prepare dialysis solutions for various dialysis application, including peritoneal dialysis solutions and hemodialysis solutions for use in acute and chronic therapies.

The expressions “raw water” and “tap water” are interchangeably used herein. The expression “raw water” generally means water that has not been specifically treated or purified and does not have any of its minerals, ions, particles, bacteria removed. Raw water includes, for example, rainwater, ground water, and water from lakes or rivers. In the context of the present invention, the expression “raw water” specifically also includes water as described before which has undergone some treatment, including, for example, tap water or bottled water. The expression “tap water” refers to water which is supplied to a tap. Tap water can be raw water, from controlled sources, which has not been purified, distilled or otherwise treated. In the context of the present invention “tap water” also refers to water which has undergone sanitary engineering, such as in water plants, before it is supplied to households. As such, tap water can also be referred to as potable water.

It was found that the osmotic dilution by harvesting the high osmotic pressure of the dialysis concentrate using tap water is feasible and highly effective. Concerns regarding loss of ions from the concentrate to the feed as well as ion diffusion from the tap water to the diluted dialysate can be sufficiently addressed with devices and methods according to the invention. Commercial CTA membranes and tailor-made thin film composite FO hollow fiber membranes (HFTFC) were selected as a means for analyzing the possibility of using osmotic dilution in hemodialysis.

It was one object of the present invention to provide hollow fiber membrane modules for generating ready-to-use dialysis fluid from a liquid dialysis concentrate by forward osmosis. It was found that highly efficient, easy-to-prepare hollow fiber filter modules for use in forward osmosis for the generation of ready-to-use dialysis fluid can be prepared from generally available dialysis membranes and hemodialyzers based on polysulfone-type hydrophobic polymers and hydrophilic polymers, such as, for example, PVP.

According to one aspect of the invention, dialysis membranes which are currently being used in hemodialyzers for the extracorporeal treatment of blood in hemodialysis can be used for the preparation of forward osmosis hollow fiber membranes and filter modules comprising same.

According to one aspect of the present invention, a hollow fiber membrane device for the generation of ready-to-use dialysis fluid for use in dialysis by forward osmosis comprises a bundle of hollow fiber membranes prepared from a first hydrophobic polymer selected from polysulfone, polyethersulfone or poly(aryl)ethersulfone and a second polymer which is polyvinylpyrrolidone (PVP), and which have a polyamide layer on the lumen side of the hollow fibers. Such devices can be advantageously prepared from so-called high-flux hemodialyzers as they are currently used for hemodialysis treatment in chronic hemodialysis. The polyamide layer can be applied to otherwise ready-to-use commercial hollow fiber membranes or hemodialyzers comprising such hollow fiber membranes.

According to one aspect of the invention, hollow fiber membrane devices according to the invention can be prepared from hollow fiber membranes comprising PVP and polysulfone and hemodialyzers comprising same. All polysulfone-based dialysis membranes possess a foam-like support structure that is designed to achieve specific separation characteristics. The increased hydraulic resistance of a foam-like support structure is partially compensated for by a reduction in wall thickness. Examples for this type of membranes, are, for example, Helixone membranes from Fresenius Medical Care. According to another aspect of the present invention, polyethersulfone/PVP/polyamide membranes are used. The so-called Polyamix membrane has a unique asymmetric, three-layer structure in which the outer layer, referred to as the supporting layer, is characterized by a very open finger-like morphology. The actual inner separation layer of the membrane consists of an extremely thin inner skin supported by an intermediate layer. This middle layer forms a foam-like structure that is very permeable. Thus, low resistance for convection and diffusion is ensured. The outer layer provides high mechanical stability.

According to yet another aspect of the present invention, hollow fiber membrane devices according to the invention can be prepared from hollow fiber membranes comprising polyethersulfone (PES) or poly(aryl)ethersulfone (PAES) and PVP, and hemodialyzers comprising same. Most membranes made of PES or PEAS and PVP are characterized by their asymmetric structure, a dense selective inner skin, which usually is in contact with blood and, in the present case, with raw water, and a supportive porous outer layer. By appropriately adjusting the membrane-manufacturing parameters, as well as using PVP of different molecular weights, the underlying membranes' physicochemical properties, morphological structure, solute-rejection behavior, and filtration performance can be adjusted appropriately. The manufacture of polysulfone/PVP based membranes, including those prepared from PES or PAES, is comparable to the production of other hollow fiber membranes and is basically known in the art (see, for example, Boschetti-de-Fierro et al., Membrane Innovation in Dialysis, Ronco (ed): Expanded Hemodialysis—Innovative Clinical Approach in Dialysis. Contrib Nephrol. Basel, Karger, 2017, vol 191, pp 100-114). For optimized applications, the inner diameter of a hollow fiber membrane ranges from 170 to 220 μm. Synthetic polymeric membranes have a wall thickness of between 25 and 55 μm.

Polysulfone/PVP based membranes, including those prepared from PES or PAES, which can be used according to the invention, can be classified as “high-flux” (HF) membranes. These membranes have been described in the literature. Today, diffusion-induced phase separation processes are primarily used, which permit polymer combinations and the fine-tuning of pore size and diffusive-transport characteristics. The polymers are dissolved in a suitable solvent, and precipitation takes place in a non-solvent bath, preferably water. The concentration of the polymer in the polymer solution is approximately 20 wt %, depending on the particular recipe. The polymer solution is pumped through an annular die (spinneret) to form a hollow fiber. The inner void of the hollow fiber is formed by a bore liquid (a mixture of solvent and non-solvent), which is introduced into the inner part of the spinneret. In a third step, the hollow fiber is guided through a non-solvent bath. The non-solvent bath and bore liquid are required to convert the homogeneous liquid-polymer solution into a two-phase system via diffusive solvent/non-solvent exchange (immersion precipitation). The demixing process stops at the vitrification point of the polymer-rich phase. A rigid membrane structure is formed during the polymer-rich phase, and the membrane pores are formed during the liquid-polymer-poor phase. The major influences on membrane properties during the manufacturing process are composition, viscosity and temperature of the polymer solution, the use of additives, the ability to crystallize or aggregate, nozzle design, composition of the coagulation bath, the conditions between the nozzle and coagulation-bath entrance, specifically the temperature and the humidity in the spinning shaft, and potentially also finishing treatments such as drying and or sterilizing the membrane with heat or by irradiation. (see, for example, Carina Zweigart, Adriana Boschetti-de-Fierro, Markus Neubauer*, Markus Storr, Torsten Boehler, Bernd Krause (2017) 4.11 Progress in the Development of Membranes for Kidney-Replacement Therapy. In: Drioli, E., Giorno, L., and Fontananova, E. (eds.), Comprehensive Membrane Science and Engineering, second edition. vol. 4, pp. 214¬247. Oxford: Elsevier). The performance of the final fiber bundle and filter are otherwise influenced by undulation of the fibers, which provides them with a wavy geometry, as described in EP 3 010 629 A1. The final assembly of a filter is described in Zweigart et al., 2017. Membranes and filters according to the invention can be sterilized by several, generally known means. It will be advantageous to sterilize devices according to the invention by irradiation with gamma rays or e-beam, all of which are standard techniques. The radiation dose for gamma ray sterilization is between 5 and 40 kGy. Steam sterilization can also be used and is the method of choice in terms of environmental impact and patient application.

According to one aspect of the invention, the hollow fiber filtration device for the generation of ready-to-use dialysis fluid by forward osmosis comprises high-flux membranes. High-flux membranes are used in devices, such as, for example, Polyflux® 170H (Baxter), Revaclear® (Baxter), Ultraflux®EMIC2 (Fresenius Medical Care), or Optiflux® F180NR (Fresenius Medical Care) and have been on the market for several years. Methods for their production have been described, for example, in U.S. Pat. No. 5,891,338 and EP 2 113 298 A1. In polysulfone or polyethersulfone based support membranes as referred to in this application, the polymer solution generally comprises between 10 and 20 weight-% of polyethersulfone or polysulfone as hydrophobic polymer and 2 to 11 weight-% of a hydrophilic polymer, in most cases PVP, wherein said PVP generally consists of a low and a high molecular PVP component. The resulting high-flux type membranes generally consist of 80-99% by weight of said hydrophobic polymer and 1-20% by weight of said hydrophilic polymer. During production of the membrane the temperature of the spinneret generally is in the range of from 25-55° C.

Polymer combinations, process parameters and performance data can otherwise be retrieved from the references mentioned or can be taken from publicly available data sheets. The expression “high-flux membrane (s)” as used herein otherwise refers to membranes having a MWRO between 5 kDa and 10 kDa and a MWCO between 25 kDa and 65 kDa, as determined by dextran sieving measurements according to Boschetti-de-Fierro A et al., Extended characterization of a new class of membranes for blood purification: The high cut-off membranes. Int J Artif Organs 2013; 36(7), 455-463.

Composite hollow fiber membranes according to the invention, which are characterized by a thin polyamide layer on the lumen side of the hollow fiber support membrane described above, are prepared from the before described hollow fibers and/or devices comprising same following generally known processes. The process of interfacial polymerization as such is known in the art and comprises contacting the interior (lumen) side of the hollow fiber support membranes with an aqueous m-phenylenediamine (MPD) solution, thereby creating an aqueous MPD layer on the lumen of the fibers. In a next step, a trimesoyl chloride (TMC)-hexane solution is passed through the lumen of the hollow fibers, and a polymerization reaction between MPD and TMC is started under suitable conditions, thereby forming a thin (nm range) polyamide layer on the lumen of the support membrane. This process of interfacial polymerization for the production of thin-film composite membranes is described in, for example, Verissimo et al, J Mem Sci 264 (2005) 48-55, for reverse osmosis hollow fiber membranes.

According to the present invention, the process was optimized in that after introduction of an MPD solution, which is allowed to remain within the fibers for a given time, the fibers are blow-dried with an inert gas, for example with compressed nitrogen gas, before the TMC solution is pumped into the lumen of the fibers. This step allows to obtain a stable, smooth and even surface and avoids an excess of MPD in the fiber before passing the TMC solution through the fiber, which can lead to the formation of an unstable film prone to collapse or be otherwise defective. It was found that the process including such blow-drying positively affects the average permeate flow rate of the modules.

According to one aspect of the invention, a process for preparing a FO membrane by interfacial polymerization comprises the steps of
(a) contacting the interior (lumen) side of a hollow fiber support membrane with an aqueous m-phenylenediamine (MPD) solution;
(b) allowing the MPD solution to contact the surface for 1 to 4 minutes;
(c) removing excess MPD solution by blow-drying the interior of the fibers with an inert gas;
(d) contacting the interior side of the hollow fiber membranes with a TMC-hexane solution;
(e) allowing the TMC-hexane solution to contact the surface for 0.5 to 2 minutes;
(f) circulating hot water with a temperature of from 85° C. to 95° C. in the lumen of the hollow fiber membranes for 3 to 10 min to cure the nascent polyamide layer;
(g) optionally storing the hollow fiber membranes in deionized water before further use.

Alternatively, step (f) comprises storing the hollow fiber membranes or hollow fiber membrane module in an oven at 100° C., wherein the temperature can be varied within a range of ±20° C. depending on the sensitivity of the material against heat.

The concentration of the MPD in the aqueous solution can be varied over a wider range. According to one aspect of the present invention, the concentration is from 0.5 to 5.0 wt.-%. According to another aspect of the invention the concentration is from 1.0 to 3.0 wt.-%. According to yet another aspect of the present invention, the concentration is 2.0 wt.-%.

The concentration of TMC in hexane can also be varied. According to one aspect of the invention, the concentration is from 0.08 to 1.0 wt.-%. According to another aspect of the invention the concentration is from 0.1 to 0.5 wt.-%.

According to yet another aspect of the present invention, the concentration is 0.1 to 0.2 wt.-%.

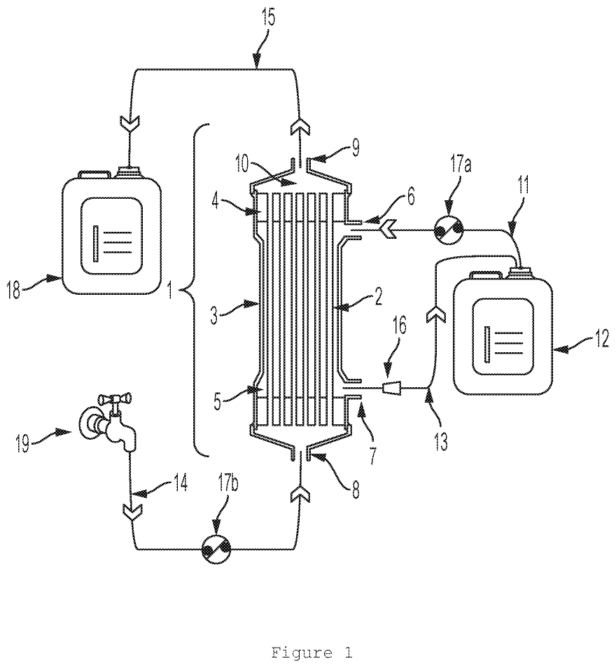

The above process is advantageously applied to filter modules which can then immediately be used for forward osmosis according to the invention. However, it is of course also possible to use isolated hollow fibers or fiber bundles which after interfacial polymerization as described before are used for the assembly of a final filtration device. Said composite hollow fiber membranes and modules can be used for the generation of dialysis fluid from dialysis concentrate from raw or tap water by forward osmosis. A module for such generation of dialysis fluid is schematically shown in FIG. 1.

It was found that the composite hollow fiber membranes and filtration devices according to the invention provide for an excellent permeability for pure water measured in L/m² hbar, see Example 1 and Table 2. Accordingly, the FO membrane according to the invention can be characterized by a water permeability of between 1.0 L/m² hbar and 1.6 L/m² hbar. According to one embodiment of the invention, the FO membrane is characterized by a water permeability of between 1.2 L/m² hbar and 1.5 L/m² hbar, specifically by a water permeability of between 1.25 L/m² hbar and 1.40 L/m² hbar.

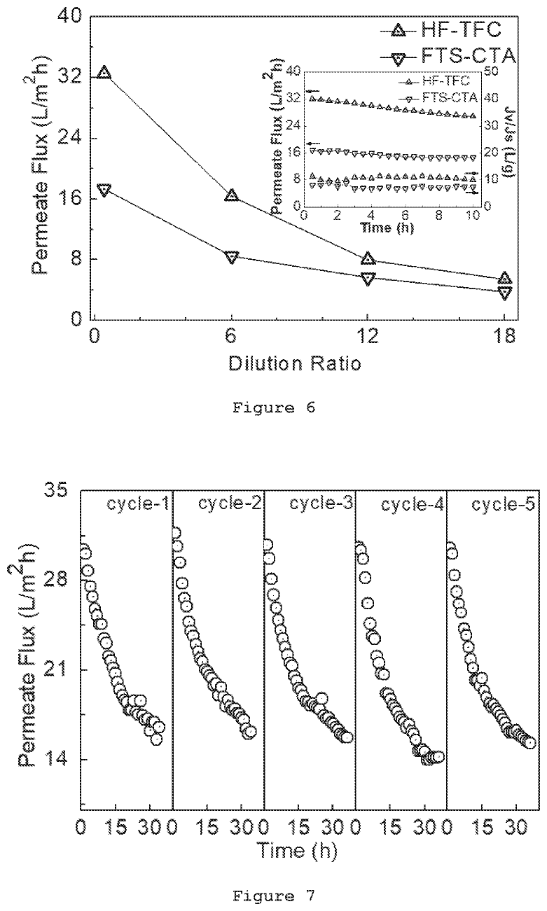

The composite hollow fiber FO membrane according to the invention is further characterized by an excellent “permeate flow” or “permeate flux” (Jᵥ) of between 10.0 L/m² h and 20.0 L/m² h. According to one embodiment of the invention, the FO membrane is characterized by a permeate flow (Jᵥ) of between 12.0 L/m² h and 18.0 L/m² h, specifically by a permeate flow (Jᵥ) of between 14.0 L/m² h and 16.0 L/m² h. As can be seen from Example 3 and FIG. 6, the permeate flux or “FO flux” declines as a function of the dilution ratio. In other words, the permeate flux decreases as the dilution ratio (DR) increases. As can be seen in FIG. 6, the permeate flux remains sufficiently high for achieving relevant dilution ratios in a process as proposed herein.

FIG. 7 gives additional details on said dependency.

According to one aspect of the present invention, a hollow fiber membrane filtration device (1) for the generation of ready-to-use dialysis fluid for use in hemodialysis by forward osmosis, comprises a plurality of hollow fibers (2) axially extending through a cylindrical housing (3) and being embedded and held, at their open ends, in a molding compound (4), thereby isolating said hollow fibers from a first fluid chamber (5) which is defined by the outer surface of said hollow fibers and the inner surface of said housing, wherein said first fluid chamber has an first fluid inlet (6) and a first fluid outlet (7), both provided on the housing, and further comprising a second fluid inlet (8) and a second fluid outlet (9) which are in communication with a second fluid chamber (10) which is defined by the interior of the hollow fibers, characterized in that the hollow fibers consist of a composite membrane comprising a hollow fiber support membrane which is comprised of 80-99% by weight of at least one hydrophobic polymer selected from the group consisting of polysulfone (PS), polyethersulfone (PES) and polyarylethersulfone (PAES), and 1-20% by weight of polyvinylpyrrolidone (PVP), and a polyamide layer on the lumen side of the hollow fiber support membrane.

According to another aspect of the invention, the composite hollow fiber membrane provides for an average permeate flow rate of between 10.0 L/hm² to 20.0 L/hm².

According to yet another aspect of the invention, the composite hollow fiber membrane has a salt permeability coefficient of between 0.14 L/m² h and 0.24 L/m² h.

According to yet another aspect of the present invention, the composite hollow fiber membrane has a total wall thickness of from 27 μm to 50 μm and an inner diameter of from 170 μm to 230 μm.

According to yet another aspect of the present invention, the composite hollow fiber membrane has an asymmetric three-layer structure consisting of a dense layer on the lumen side of the hollow fiber membrane having a thickness of below 0.6 μm, followed by a support layer having a sponge structure and a thickness of from 1 to 15 μm, and a third layer having a finger-structure and a thickness of from 25 to 50 μm.

According to another aspect of the present invention, the total usable membrane surface area of a device for use according to the invention is between 1.5 and 2.8 m² and the packing density of the hollow fibers within the housing is between 45% and 70%.

According to one embodiment of the invention, the packing density of the composite hollow fiber membranes in the modules of the present invention is from 50% to 65%, i.e., the sum of the cross-sectional area of all hollow fiber membranes present in the module amounts to 50 to 65% of the cross-sectional area of the part of the housing comprising the bundle of composite hollow fiber membranes. According to one embodiment of the present invention, the packing density of the composite hollow fiber membranes in the module of the present invention is from 53% to 60%. A typical fiber bundle with fibers according to the invention, and which is located within a housing having an inner diameter of, for example, 38 mm, wherein the fibers have an effective fiber length of 236 mm and wherein packing densities of between 53% to 60% are realized, will contain about 12 500 to 13 500 fibers, providing for an effective surface area of about 1.7 m². It will be readily understood by a person skilled in the art that housing dimensions (inner diameter, effective length) will have to be adapted for achieving lower or higher membrane surface areas of a device, if fiber dimensions and packing densities remain the same.

According to another aspect of the present invention, a bundle of composite hollow fiber membranes according to the invention is present in the housing or casing, wherein the bundle comprises crimped fibers. The bundle may contain only crimped fibers, such as described, for example, in EP 1 257 333 A1. According to another aspect of the invention, the fiber bundle may consist of 80% to 95% crimped fibers and from 5% to 15% non-crimped fibers, relative to the total number of fibers in the bundle, for instance, from 86 to 94% crimped fibers and from 6 to 14% non-crimped fibers. In one embodiment, the proportion of crimped fibers is from 86 to 92%. The fibers have a sinusoidal texture with a wavelength in the range of from 6 to 9 mm, for instance, 7 to 8 mm; and an amplitude in the range of from 0.1 to 0.5 mm; for instance, 0.2 to 0.4 mm. Incorporation of 5 to 15% non-crimped fibers into a bundle of crimped semi-permeable hollow fiber membranes may enhance the performance of the FO filtration device of the invention.

As mentioned before, the fiber bundle is comprised of a number of hollow fiber membranes that are oriented parallel to each other. The fiber bundle is encapsulated at each end of the dialyzer in a potting material to provide for a first flow space surrounding the membranes on the outside and a second flow space formed by the fiber cavities and the flow space above and below said potting material which is in flow communication with said fiber cavities. The filtration device generally further consists of end caps capping the mouths of the tubular section of the device which also contains the fiber bundle. The body of the device also includes an inlet and an outlet for the dialysis concentrate which is diluted during the process of dialysis fluid generation until the target concentration has been achieved. They can therefore also be addressed as “dialysate inlets” and “dialysate outlets”.

According to one embodiment of the invention, the dialysate inlet and dialysate outlet define fluid flow channels that are in a radial direction, i.e., perpendicular to the fluid flow path of the tap or raw water. The dialysate inlet and dialysate outlet are designed to allow liquid dialysis concentrate or dialysis fluid to flow into an interior of the dialyzer, bathing the exterior surfaces of the fibers and the fiber bundle, and then to leave the dialyzer through the outlet. The membranes allow raw water to flow therethrough in one direction with liquid dialysis concentrate or dialysis fluid flowing on the outside of the membranes in opposite direction. Pure water is thereby passing the membrane from the lumen side in the direction of the concentrate on the outer side of the membranes. By doing so, the concentrate becomes more and more diluted.

A variety of designs can be utilized for accomplishing the present invention. According to one embodiment the hemodialyzers of the invention have designs such as those set forth in WO 2013/190022 A1. However, other designs can also be utilized.

According to one aspect of the invention, the method of preparing ready-to-use dialysis fluid from raw water by forward osmosis, comprises the steps of
a) providing a hollow fiber membrane filtration device comprising a plurality of forward osmosis hollow fiber membranes (2) axially extending through a cylindrical housing (3) and being embedded and held, at their open ends, in a moulding compound (4), thereby isolating said hollow fibers from a first fluid chamber (5) which is defined by the outer surface of said hollow fibers and the inner surface of said housing, wherein said first fluid chamber has an first fluid inlet (6) and a first fluid outlet (7), both provided on the housing, and further comprising a second fluid inlet (8) and a second fluid outlet (9) which are in communication with a second fluid chamber (10) which is defined by the interior of the hollow fibers;
b) passing raw water through the second fluid chamber (10) of the filter device, while recirculating a liquid dialysis concentrate through the first fluid chamber (5) in a countercurrent, thereby creating a flux of pure water from the lumen side of the composite hollow fiber membrane into the dialysis concentrate;
c) terminating recirculation upon reaching the target concentration of the dialysis solution; and
d) providing the ready-to-use dialysis fluid for use in an extracorporeal hemodialysis circuit.

According to another aspect of the invention, the hollow fiber membrane filtration device used according to step (a) in the method for preparing ready-to-use dialysis fluid is a thin-film composite (HF-TFC) membrane prepared from a hollow fiber support membrane which is comprised of 80-99% by weight of at least one hydrophobic polymer selected from the group consisting of polysulfone (PS), polyethersulfone (PES) and polyarylethersulfone (PAES), and 1-20% by weight of polyvinylpyrrolidone (PVP), and a polyamide layer on the lumen side of the hollow fiber support membrane.

According to one further aspect of the invention, the hollow fiber membrane filtration device used according o step (a) in the method for preparing ready-to-use dialysis fluid is a forward osmosis membrane selected from the group consisting of cellulose acetate (CA) membranes, cellulose triacetate (CTA) membranes, thin film composite (TFC) membranes, and bio-mimetic membranes.

CA and CTA membranes are commercially available and can be purchased, for example, from Fluid Technology Solutions, Inc. (FTS). Thin film composite membranes are also known in the art. The support layer of TFC membranes is generally made from polyethersulfone or polysulfone, onto which a thin (around 200 nm) polymeric rejection layer is formed on top of the support membrane by interfacial polymerization of m-phenylenediamine (MPD) and trimesoyl chloride (TMC). A similar process has been used since more than two decades to produce RO membranes. The difference between TFC FO and TFC RO membranes lies mainly in the support substrate, which for FO membranes is preferably more porous, more hydrophilic, and thinner. Modified variants of polyethersulfone or polysulfone based support membranes have been described which, for example, comprise TiO₂, SiO₂, or graphene oxide (GO). See, for example, Sirinupong et al, Arabian Journal of Chemistry (2018) 11, 1144-1153. Sulfonated polysulfone (SPSU)/poly(vinyl chloride) (PVC) substrates have also been used for the production of TFC membranes for FO (Zheng et al, Scientific Reports (2018) 8, 10022. Biomimetic membranes which can be used according to the invention generally comprise the aquaporin water channel protein and are commercially available, for example from Aquaporin, such as the HFFO2 or HFFO6 hollow fiber modules. For example, see also WO 2015/124716 A1.

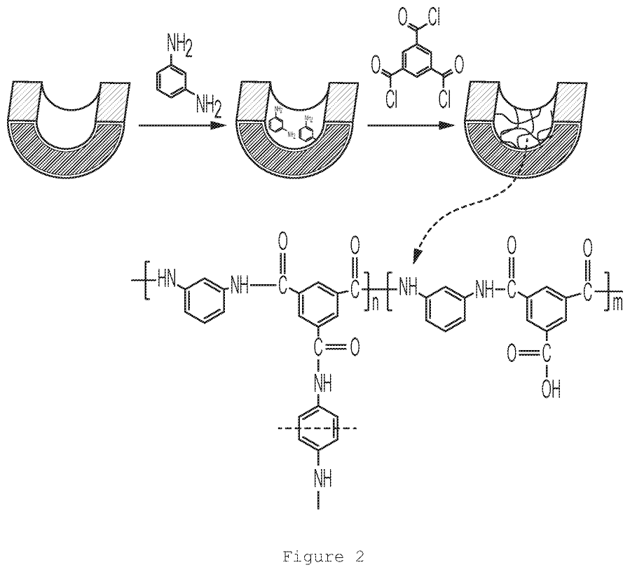

According to another aspect of the invention, it is possible to use several FO filtration devices according to the invention in series or in parallel configuration in a setup according to FIG. 1, which serves to increase the overall throughput, thereby allowing an increase in the amount of dialysis fluid per time. According to one embodiment of the invention, 2 to 5 hollow fiber filtration devices are connected in parallel, or 2 to 3 hollow fiber filtration devices are connected in series. For example, where two filtration devices are connected in parallel, the tap water and dialysis solution delivery system and respective outlet lines are connected to the hollow fiber filtration device via adaptors comprised of flexible tubing(s) in the shape of a “Y”. Operating filtration devices in parallel is generally known in the art. A possible setup is shown, for example for hemodialyzers, in FIG. 2 of Hootkins, Dialysis & Transplantation September 2011, 392-396.

In order to determine when the target concentration of the dialysis solution is reached and the process can be stopped, the system can either be calibrated for a given dialysis concentrate and volume of said concentrate. Alternatively, the solution of this problem is attained according to the claimed features of the invention, and by providing at least one electrolyte detector in the return line connected to the outlet of the first fluid chamber of the hollow fiber membrane filtration device and, optionally, a second electrolyte detector in the dialysis concentrate feed line connected to the inlet of the first fluid chamber of the hollow fiber membrane filtration device, each detector coupled to a readout element of the controlling unit through which both of the values of the dialysis solution can be observed and eventually controlled. The concentration of the dialysis fluid determined with an electrolyte detector in the return line connected to the outlet of the first fluid chamber of the hollow fiber membrane filtration device provides that the composition of the dialysis solution can be suitably controlled and accurately adjusted to the preset dialysis fluid concentration, thereby also determining the termination of the process for preparing ready-to-use dialysis fluid.

According to a first embodiment, a control unit is provided in combination with the above-mentioned at least one electrolyte detector. In another embodiment, the control unit is provided in combination with two detectors in an attached differential unit, i.e., a comparator, which can indicate the difference of the composition of the electrolyte contents before and after the hollow fiber membrane filtration device differentially. Thus, the difference of the electrolyte contents of fluids circulating through the filtration device can be controlled. The electrolyte detectors can be connected to an actual value device which, in turn, is connected with a preprogrammed reference value device. If the actual value deviates from the reference, which is the final ready-to-use dialysis fluid concentration, the composition is corrected by further recirculation of the dialysis concentrate until the actual value coincides with the reference value.

In order to determine the total ion concentration, either the conductivity measurement or the determination of ion potentials, particularly sodium ions, can be advantageously carried out by means of ion-selective electrodes. The latter method has the advantage over the former that several types of ions can be measured selectively and adjusted with the aid of the device according to this invention. On the other hand, the electrodes applied are much more unstable and breakable than the ion-conductivity cell, so that the conductivity measurements are preferred for the method and system according to the invention.

According to the invention, the type of liquid concentrate, often also referred to as dialysate precursor composition, used in the before described processes is not restricted to specific concentrates. Obviously, the concentrates used for generating dialysis fluids should have a composition which is suitable for dialysis fluids which can be used, for example, in PD or HD therapies.

According to one aspect of the invention, dialysis fluid is prepared for use in hemodialysis. In modern dialysis machines, dialysate for hemodialysis is made by mixing two concentrate components, which may be provided as liquid or dry (powder) concentrates, with pure water from the RO plant of the clinics. According to the present invention the same concentrates can be used to prepare dialysis fluids where no such RO water is available. The said two concentrate components generally comprise bicarbonate on the one hand and an acid concentrate on the other hand. The bicarbonate component contains sodium bicarbonate and sodium chloride; the acid component contains chloride salts of sodium, potassium (if needed), calcium, magnesium, acetate (or citrate), and glucose (optional). The relative amounts of water, bicarbonate, and acid components define the final dialysate composition. Bicarbonate has replaced acetate as the dialysate buffer in most countries. Typical concentrations of dialysate components are given in below Table A, see Kotanko et al., Comprehensive Clinical Nehrology (4^{th} Edition), 2010, pages 1053-1059.

TABLE A
Composition of dialysis solutions for bicarbonate dialysis

| | Concentration | Typical |
|---|---|---|
| Component | Range | Concentration |
| Electrolytes (mmol/l) | | |
| Sodium | 135-145 | 140 |
| Potassium | 0-4.0 | 2.0 |
| Calcium | 0-2.0 | 1.25 |
| Magnesium | 0.5-1.0 | 0.75 |
| Chloride | 87-124 | 105 |
| Buffers (mmol/l) | | |
| Acetate | 2-4 | 3 |
| Bicarbonate | 20-40 | 35 |
| pH | 7.1-7.3 | 7.2 |
| pCO₂ (mmHg) | 40-100 | |
| Glucose | 0-11 (0-200 mg/dl) | 5.5 (100 mg/dl) |

Dialysate containing citrate has been introduced with a view on reduction in heparin dose.

Most dialysis fluids contain one or several substances, in varying compositions and concentrations, chosen from the group consisting of glucose (including icodextrin), bicarbonate, potassium, acetate, lactate, citrate, magnesium, calcium, sodium, sulfate, phosphate and chloride. The concentrates also contain water. The composition and target concentrations of dialysis solutions for use in PD or HD are generally known in the art.

According to one aspect of the present invention, commercially available dialysis concentrates can be used for preparing ready-to-use dialysis fluids.

According to one specific embodiment of the invention, the concentrate is a citric acid concentrate liquid which, for example, can be used together with a bicarbonate based solution in hemodialysis as described above. Such citric acid concentrate liquids contain, for example, citric acid, magnesium, calcium, potassium, sodium and optionally also dextrose. Examples for such concentrates are CitraPure Liquid Acid Concentrate (Baxter) or Citrasate Liquid Acid Concentrate (Fresenius Medical Care).

According to another embodiment of the present invention, bicarbonate based concentrate liquids can be used. Such concentrates contain, for examples, sodium bicarbonate. Examples for such concentrates are SteriLyte Liquid Bicarbonate (Baxter).

According to yet another embodiment of the present invention, acetic acid liquids can be used. Such concentrates contain, for example, acetic acid, magnesium, calcium, potassium, sodium and optionally also dextrose. Examples for such concentrates are RenalPure Liquid Acid Concentrate (Baxter) or NaturaLyte (Fresenius Medical Care).

According to another aspect of the invention, dialysis fluid is prepared for use in peritoneal dialysis. The peritoneal dialysis fluids have traditionally been provided in bags, often as 1.5 L, 2 L, 3 L, 5 L, or 6 L bags, and being terminally sterilized. Shipping and storage of the sheer volume of fluids required is both tremendously inconvenient and expensive. A review on current PD solutions is available from Garcia-Lopez et al., Nat. Rev. Nephrol. 8 (2012), 224-233. In general, standard peritoneal dialysis fluids contain glucose at a concentration of 1.5%-4.25% by weight to effect transport of water and metabolic waste across the peritoneal membrane. Glucose is generally recognized as a safe and effective osmotic agent, particularly for short dwell exchanges. Newer PD solutions contain alternatives to glucose, namely icodextrin such as in Extraneal (Baxter) or amino acids such as in Nutrineal (Baxter). Most solutions contain glucose-based solutions buffered either with lactate, bicarbonate and lactate, or bicarbonate; some are provided in single chamber (e.g. Dianeal (Baxter), Extraneal, Nutrineal, Stay-safe (Fresenius Medical Care)) whereas others are provided in multicompartment bag systems to separate the buffer from the glucose compartment (e.g. Physioneal (Baxter), Nicopeliq (Terumo) or Balance (Fresenius Medical Care)).

As disclosed in EP 3 452 136 A1 and EP 452 139 A1, PD solutions can also be prepared from liquid concentrates, wherein ready-to-use dialysis fluid is prepared by mixing at least a first and a second concentrate with water. The approach described in said reference differs from the presently described approach in how the ready-to-use dialysis fluid is generated. However, the same or equal concentrates can be used in the method and system described herein. For example, the concentrates used according to the invention, may comprise a first concentrate comprising glucose which has a pH of between 1.5 and 4 or a pH of between 2 and 3.5 or a pH between 2.2 and 3.0; and a second concentrate comprising a physiologically acceptable buffer which has a pH of between 6.0 and 8.5.

The prepared ready to use peritoneal dialysis fluid may then have the following content:

Sodium (Na+) 100-140 mM, potassium (K+) 0-4 mM, calcium (Ca2+) 0-2 mM, magnesium (Mg2+) 0-0.75 mM, lactate, bicarbonate, glucose.

According to one embodiment of the invention the physiologically acceptable buffer is selected from the group consisting of acetate, lactate, citrate, pyruvate, carbonate, bicarbonate, and amino acid buffer; or mixtures thereof.

According to another embodiment of the invention said first concentrate further comprises at least one electrolyte selected from the group consisting of sodium, calcium, magnesium, and optionally potassium. According to another embodiment of the invention, said second concentrate further comprises at least one electrolyte selected from the group consisting of sodium, calcium, magnesium, and optionally potassium.

According to another embodiment of the invention said further concentrate comprises at least one of electrolyte selected from the group comprising sodium, calcium, magnesium, and optionally potassium. In another embodiment of the invention, said further concentrate comprises a physiologically acceptable buffer selected from the group comprising acetate, lactate, citrate, pyruvate, carbonate, bicarbonate, and amino acid buffer; or mixtures thereof.

### EXAMPLES

### 1. Materials and Methods

### 1.1 Chemicals and Materials

A commercial flat sheet cellulose triacetate membrane (CTA) prepared from cellulose triacetate was used for comparison. Hollow fiber thin-film composite (HF-TFC) membranes according to the invention were prepared as schematically described in FIG. 2, starting from commercially available hollow fiber membranes based on polyethersulfone and PVP (polyvinylpyrrolidone), available from Baxter under the trade name Revaclear.

For preparing the HF-TFC, an aqueous solution consisting of MPD and H₂O was prepared with an MPD concentration of 2 wt.-%. This concentration can be varied over a wider range. The concentration used here was found to be efficient. Other surfactants and/or bases can be added for modifying the reaction.

The aqueous phase was slowly pumped into the lumen of the fibers for a contact time of 3 minutes. After removing the aqueous phase, the inner surfaces were blow-dried using compressed nitrogen gas. Subsequently, the TMC-hexane solution (0.15 wt %) was supplied to the hollow fiber membrane lumen allowing for a reaction time of 1 min. Then hot water with a temperature of 85° C. (can vary from 85-95° C.) was circulated in the lumen of the hollow fiber membrane module for 5 min to cure the nascent polyamide layer. Alternatively, the module can be stored in an oven at 100° C., wherein the temperature can be varied within a range of from ±10 to 20° C. depending on the sensitivity of the material against heat. Finally, the TFC hollow fiber membrane module was stored in DI water before further characterization.

Dialysis concentrate (Select Bag One AX250G) was kindly supplied by Baxter Co. Ltd (Suzhou) and tap water was supplied by Shanghai Waterworks. Deionized (DI) water of conductivity in the range of 7-15 μS/cm was used in the experiment.

### 1.2 RO Test and Modeling of Ion Rejections

The water permeability coefficient (A value) and salt permeability coefficient (B value) were quantified in the cross-flow RO filtration system as described in the literature. See, for example, Cath et al., Desalination 312 (2013) 31-38.

In addition, the membrane structural parameter S and other involved parameters were also determined using the standard protocol described before.

### 1.3 Characterization of FO Membranes

A bench-scale FO test setup was used for membrane characterization as described before (see, for example, Cath et al., 2013). For flat sheet FO membrane, the effective area of the membrane cell was 24 cm² (i.e., length, width and height were 8, 3, and 0.2 cm, respectively), the cross-flow rates of the feed and draw solutions were monitored with rotameters and kept constant at 0.9 L/min (or a flow velocity of 0.25 m/s). For the hollow fiber TFC membrane, a module that has 5 fibers (an effective surface area of 24.2 cm²) was used for the test, the cross-flow rates of the feed and draw solutions were monitored with rotameters and kept constant at 0.2 L/min and 1.8 L/min, respectively. Otherwise, characterization conditions were consistent with the flat sheet membrane.

For characterizing the FO membranes, the initial volume of both feed and draw solutions were 2.0 L DI water and 0.5 M NaCl, respectively. An electronic balance (CP2002, Ohaus Instrument Co., Ltd.) connected to a computer recorded the weight increase of water which permeated into the draw solution. The FO water flux was measured by monitoring the change in the weight of the draw solution, and the reverse salt flux was calculated based on the conductivity change in the feed.

If not mentioned otherwise, tests were performed for 30 minutes to evaluate the water flux and reverse salt flux. The water flux, Jᵥ, was based on a measurement over 5 min under stable conditions by using the following equation (5),

$${J}_{v} = \frac{Δ m}{{A}_{m} Δ t {ρ}_{draw}} \qquad ( 5 )$$

wherein Δm, Δt, Δₘ and ρ_{draw} represent the mass of permeation water, time interval, effective membrane surface area, and draw solution density, respectively. The change of draw solution concentration was negligible and the ratio of water permeation to the draw solution was less than 5%. The reverse salt flux, namely Jₛ, of the membrane was characterized by calculating the change of salt content in the feed solution as described in Equation 6,

$${J}_{s} = \frac{{V}_{t} {C}_{t} - {V}_{0} {C}_{0}}{{A}_{m} Δ t} \qquad ( 6 )$$

wherein C₀ and Cₜ represent feed salt concentrations at beginning and end of the test; V₀ and Vₜ are the initial and the end volume of the feed, respectively; t was the operating time of the experiment. The concentration of draw solute components that leaked into the feed solution was determined by using electrical conductivity (Thermo Fisher Scientific, Waltham, Mass.) and calibration using standard solution of each component.

### 1.4 Osmotic Dilution Using Dialysate Concentrate

The experimental temperature of the test system was maintained at 25±0.5° C. The membranes were tested in FO mode. To further quantify the retention of the salts in the concentrate by the FO membranes, the ion concentration at different dilution ratio in the draw solution was sampled and measured. During the FO dilution process, the dilution ratio (DR) was defined as in Equation (7),

$$DR = \frac{{V}_{d , t}}{{V}_{d , 0}} \qquad ( 7 )$$

wherein V_{d,0} and V_{d,t} are the initial volume of draw solution and the volume at time t of the draw solution, respectively.

### 1.5 Characterization of Water and Dialysis Concentrate

Tap water quality was characterized in terms of conductivity (Mettler Toledo (LE703) conductivity meter), pH (Sartorius PB-10), and turbidity (Hach turbidity meter 2100Q). A Shimadzu inductively coupled plasma atomic emission spectroscopy (ICP-AES, ICPE-9000, Shimadzu, Kyoto) was utilized to measure the cation concentration. An ion selective liquid chromatograph (IC, LC20AT, Shimadzu, Kyoto) was used to analyze the anions. Chemical oxygen demand (COD) was determined by digestive degradation and measured by spectrophotometer (Hach DIt2800). Total organic carbon (TOC) was measured using a TOC analyzer (TOC-LCPH, Shimadzu, Kyoto).

The relevant results for tap water and dialysis concentrate are shown in Table 1, where the parameters represent an arithmetic average between three measurements collected during the experimental evaluation.

TABLE 1
Characteristics of tap water and dialysis concentrate

| Tap water (average) | | | Dialysis concentrate | | |
|---|---|---|---|---|---|
| Components | Concentration | | Components | Concentration | |
| COD | 0.4 | mg/L | K⁺ | 70 | (mmol/L) |
| TOC | 1.35 | mg/L | Ca²⁺ | 43.75 | (mmol/L) |
| Conductivity | 352.4 | μS/cm | Na⁺ | 3605 | (mmol/L) |
| Turbidity | 0.35 | NTU | Mg²⁺ | 17.58 | (mmol/L) |
| pH | 7.3 | | Cl⁻ | 3736.3 | (mmol/L) |
| K⁺ | 1.4 | mg/L | Acetic acid | 105 | (mmol/L) |
| Ca²⁺ | 36.7 | mg/L | Osmotic | 222.5 | (bar) |
| | | | pressure* | | |
| Na⁺ | 5.5 | mg/L | | | |
| Mg²⁺ | 2.8 | mg/L | | | |
| Cl⁻ | 41.7 | mg/L | | | |

### 1.6 Membrane Morphology

The morphologies of the top and bottom membrane surfaces and the cross-section were characterized by scanning electron microscopy (HITACHI TM-1000). The cross-section samples were prepared by cryogenic breaking of a wet sample in liquid nitrogen. All the membrane samples were coated with an ultra-thin layer of gold layer before measurement.

### 2. Examples

### 2.1 Characterization of the FO Membranes and Modules

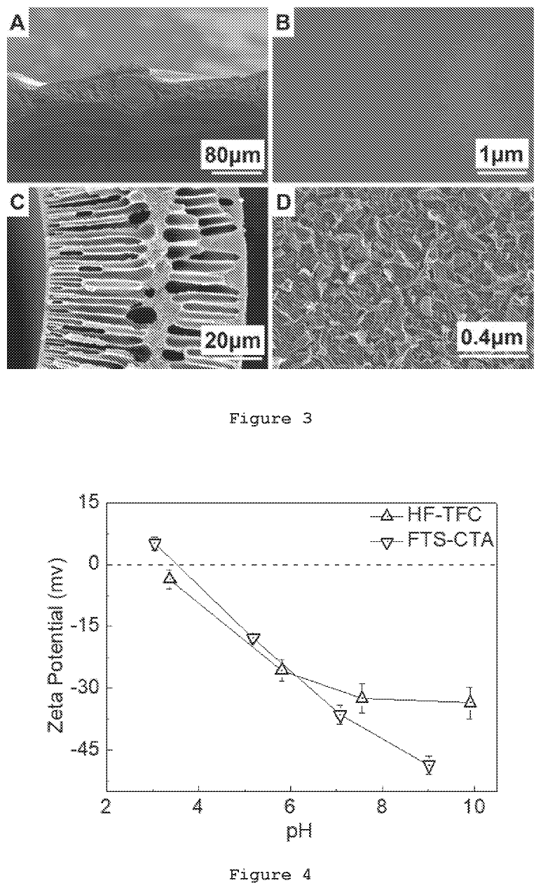

The surface and the cross-section microstructure images of CTA and TFC membranes are shown in FIG. 3. The CTA membrane has a smooth surface (FIG. 3B) and is reinforced by an embedded mesh. The composite hollow fiber HF-TFC membrane has an inner polyamide active layer prepared by interfacial polymerization; the inner surface shows rig-and-valley surface on top of a Polyethersulfone ultrafiltration support (FIG. 3D); the typical finger-like voids in the middle and sponge porous structure at the top and bottom allow fast water diffusion across the support and high water flux (FIG. 3C). Surface charges of both membranes are shown in FIG. 4. Within the pH range of 3-10, the surface of the HFTFC membranes show negative charges, and the CTA membrane shows positive charge at pH=3, but negative charges were observed at higher pH. Zeta potentials of membrane surface depend on the solution (e.g. pH and ionic strength) and the membrane polymeric chemistry. The negative charges of the TFC membrane can be attributed to the dissociation of free or uncross-linked carboxylic functional groups of the polyamide active skin layer and the adsorption of negatively charged ions. By contrast, the CTA membrane only contains the neutral acetyl functional groups. Thus, the negative charges are attributed to the adsorption of negatively charged ions (e.g. hydroxide or chloride) to the membrane surface. The hydroxyl groups are deprotonated at high pH, which explains the gradual decrease of zeta potential following the increase in pH. For TFC membranes, after all free carboxylic functional groups dissociated, no more negative charges are generated, thus the zeta potential is stabilized at higher pH. Nevertheless, for the current application, a neutral pH in both tap water and dialysate concentration is expected. In the dilution experiment, because of the presence of acetic acid in the draw side, the pH of the draw side varies from 2.15 to 3.43, corresponding to the original draw solution and diluted 18 times, respectively, which means that the charge on the membrane surface fluctuates around 0.

Table 2 lists the pure water and salt permeability coefficient, normal rejection to NaCl, structure parameter, flux in FO (Active layer facing draw) and reverse salt flux. The rejection of CTA membrane (97.4%) shows slightly higher values than that of the HF-TFC membrane (93.2%), which corresponds to the lower B value of membrane. In addition, the structure parameter of the CTA membrane (0.17 mm) is smaller than that of the TFC membrane (0.32 mm), which is most probably due to the very thin membrane wall (˜40 μm) (FIG. 3A). Although a higher S value of the TFC-type FO membrane would result in a more severe internal concentration polarization (ICP), the high mass transfer resistance of CTA membrane (low water permeability, namely, 0.38 L/m² h bar) results in a FO flux of 7.5 L/m² h, lower than the flux of hollow fiber TFC membrane (14.9 L/m² h).

TABLE 2
Properties of commercial CTA membrane and synthesized TFC FO membrane

| | A | R | B | S | Jᵥ | Jₛ/Jᵥ |
|---|---|---|---|---|---|---|
| Membrane | (L/m²hbar) | (%) | (L/m²h) | (mm) | (L/m²h) | (g/L) |
| CTA | 0.38 ± 0.01 | 97.4 ± 0.4 | 0.09 ± 0.01 | 0.17 ± 0.01 | 7.50 ± 1.0 | 0.15 ± 0.05 |
| HF-TFC | 1.32 ± 0.04 | 93.2 ± 0.2 | 0.18 ± 0.01 | 0.32 ± 0.02 | 14.9 ± 0.8 | 0.12 ± 0.03 |

A: pure water permeability;
R: salt rejection;
B: salt permeability coefficient;
S: structural parameter;
Jᵥ: FO flux;
Jₛ/Jᵥ: reverse salt selectivity.
The draw solution was 0.5M NaCl and the feed was deionized water.

### 2.2 Modeling the Transport of Ions in the RO Process

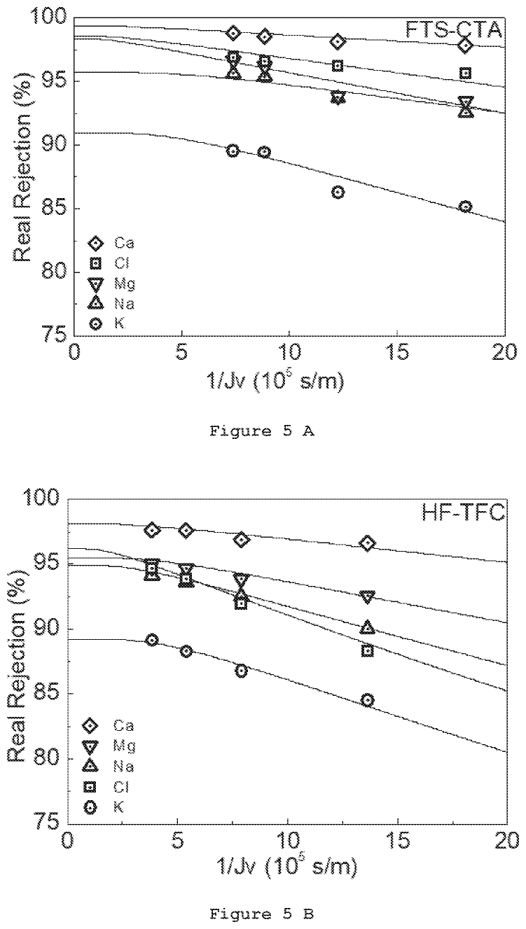

The requirement for any component for medical devices is much more stringent than industrial systems. As a preliminary technical verification experiment, the first guideline is to analyze the possibility of the salt passage across the membrane. FIG. 5 shows the real rejection of two membranes for individual ion in tap water. In general, the rejections increase with increasing flux. For ions with smaller hydration radii, the impact of flux on the rejection values appear more pronounced. Taking potassium and chloride ions as examples, both ions have relatively smaller hydrated ion radii (Table 6); as the flux increases from 2.64 to 9.35 LMH, an increase in potassium ion and chloride ion rejection from 84.5% to 89.1% and 88.3% to 94.6% for HF-TFC membrane was observed. The impact of flux on rejection of ions with larger hydration radii such as calcium and magnesium is low probably because their rejection values are high enough.

The irreversible thermodynamic model developed by Kedem and Katchalsky (Fujioka et al, 2012) was used to further elucidate the rejection behaviors of ions in RO mode. Depending on the mass transfer coefficient (k), water flux (Jᵥ) and the observed rejection data using Eq. (3), the real rejection (Rᵣₑₐₗ) at different flux was calculated using Eq. (4). The reflection coefficient (o) and solute permeability coefficient (Pₛ) were obtained by fitting the real rejection data to the irreversible thermodynamic model (Eq. 1, 2) and the data were summarized in Table 3 and Table 4. The results indicate that the solute permeability in the membrane to the major ions are different for both membranes. For CTA membrane, the order is:

K⁺ (6.14×10⁻⁸ m/s)>Mg²⁺ (3.54×10⁻⁸ m/s)˜Na⁺ (2.64×10⁻⁸ m/s)˜Cl⁻ (2.45×10⁻⁸ m/s)>Ca²⁺ (1.00×10⁻⁸).

For HF-TFC membrane, the order of solute permeability is:

K⁺ (7.80×10⁻⁸ m/s)>Cl⁻ (7.35×10⁻⁸ m/s)>Na⁺ (5.60×10⁻⁸ m/s)>Mg²⁺ (3.79×10⁻⁸ m/s)>Ca²⁺ (2.00×10⁻⁸ m/s).

Interesting observation from the permeability coefficient values were the variation in the order of both membranes. Obviously, both CTA and HF-TFC membranes showed the lowest rejection for K⁺ and highest rejection for Ca²⁺ (FIG. 5) and correspondingly highest P values. This observation fits to the hydration radii because the hydration radius of K⁺ with 0.331 nm is the smallest and that of Ca²⁺ is one of the largest (0.412 nm). The subtle difference in the rejection and P values are of no clear and solid explanation yet if only considering the steric effect. Steric effect is dominant in real rejection; Mg²⁺ has a hydration radius of 0.428 nm, which is significantly larger than Na⁺ (0.358 nm); consequently, the real rejections of Mg⁺ for both membranes are found to be higher than for Na⁺ (FIG. 5); This is true for calcium ion as well. For ions with negative charges, the Donnan effect plays a key role in rejection of anions, for example Cl⁻. Since both membranes are negatively charged (FIG. 4), both CTA and TFC membranes showed relatively high real rejection of Cl⁻ considering the relatively low rejection of K. Both K⁺ and Cl⁻ have nearly the same hydration radii (0.331-0.332 nm). Higher rejection of Cl⁻ for CTA membrane than for TFC membranes (FIG. 5) was observed which might be mainly attributed to the comparably slightly higher negative charges of CTA membranes than the TFC membrane (FIG. 4). Deviation of rejection and P values for both Ca²⁺ and Mg²⁺ are not related to both steric and Donnan effects. The Mg²⁺ of larger hydration diameter (0.428 nm) showed lower rejection and higher P value than Ca²⁺ ion (0.412 nm). This phenomenon is quite unexpected from the conventional transport models of nanofiltration. The models are based on physical characteristics of both the membrane and ions without taking considering of the chemical interaction of the ions to the membranes. It is possible that Ca²⁺ ion preferentially interact with the membrane materials. The HF-TFC membrane active layer is known to allow for the dissociation of free or uncross-linked carboxylic functional groups of the polyamide active skin layer and the adsorption of negatively charged ions. CTA membrane materials has also hydroxyl groups. It is likely that Ca²⁺ ion form chemical bonding with either carboxylic or hydroxyl functional groups of both membranes to add extra resistance to the transport of Ca²⁺ ion. Because the TFC membrane has a very thin active layer which contains the conjugative carboxylic groups to Ca²⁺, the preferential adsorption to the calcium ion is limited, leading to the similar permeability coefficient. However, in the CTA membrane, passage of the Ca²⁺ ion encounters the whole cross-section of the membrane; consequently, the adsorption of Ca²⁺ ion is significantly higher than for the thin film composite polyamide layer.

TABLE 3
Transport parameters of main ions through the CTAmembrane
and the fitting coefficient of determination (R²)
of the irreversible thermodynamic model

| CTA | k (m/s) | σ (—) | P (m/s) | R² (—) |
|---|---|---|---|---|
| K⁺ | 6.216 × 10⁻⁵ | 0.909 | 6.14 × 10⁻⁸ | 0.9998 |
| Ca²⁺ | 3.390 × 10⁻⁵ | 0.994 | 1.00 × 10⁻⁸ | 0.9992 |
| Na⁺ | 4.822 × 10⁻⁵ | 0.956 | 2.64 × 10⁻⁸ | 0.9999 |
| Mg²⁺ | 3.319 × 10⁻⁵ | 0.984 | 3.54 × 10⁻⁸ | 0.9998 |
| Cl⁻ | 6.374 × 10⁻⁵ | 0.986 | 2.45 × 10⁻⁸ | 0.9998 |

TABLE 4
Transport parameters of main ions through the HF-TFC
membrane and the fitting coefficient of determination
(R²) of the irreversible thermodynamic model.

| HF-TFC | k (m/s) | σ (—) | P (m/s) | R² (—) |
|---|---|---|---|---|
| K⁺ | 6.216 × 10⁻⁵ | 0.892 | 7.80 × 10⁻⁸ | 0.9998 |
| Ca²⁺ | 3.390 × 10⁻⁵ | 0.981 | 2.00 × 10⁻⁸ | 0.9999 |
| Na⁺ | 4.822 × 10⁻⁵ | 0.949 | 5.60 × 10⁻⁸ | 0.9999 |
| Mg²⁺ | 3.319 × 10⁻⁵ | 0.955 | 3.79 × 10⁻⁸ | 0.9999 |
| Cl⁻ | 6.374 × 10⁻⁵ | 0.962 | 7.35 × 10⁻⁸ | 0.9999 |

### 2.3 Osmotic Dilution of Dialysis Concentrate Using Tap Water

FIG. 6 shows the FO flux of the two membranes in the osmotic dilution declines as a function of the dilution ratio (DR) increases. At the initial stage, the initial flux of the TFC membrane (33.5 LMH) was nearly 2 times as that of the CTA membrane (17.6 LMH). At a dilution ratio of 18, the dialysate concentrate was diluted to 18 times, the fluxes of two membranes appeared to be very similar. Furthermore, the imbedded figure shows that very stable Jₛ/Jᵥ were observed and Jₛ/Jᵥ of CTA membrane is higher than TFC membranes, which suggests that Jₛ/Jᵥ is independent on draw solution concentration and the reverse salt selectivity is related to water permeability (A) and the salt permeability (B). This ratio is a key parameter in the design of osmotically driven membrane because the salt leakage is caused by the reverse salt diffusion. Because the reverse salt flux is related only to the membrane characteristics, one may analyze the difference of Jₛ/Jᵥ according to the experiment measurement, as listed in Table 2. CTA membrane has a much lower A value, but lower B value than TFC membrane. Because the A value of the TFC membrane is about 3.5 times as of CTA membrane, the reverse salt selectivity, namely Jᵥ/Jₛ, of TFC membrane is larger than that of CTA (note that the B value of CTA is only 2 times as that of TFC). This estimation corresponds well to the experimental measurement. We also notice that the experimental reverse salt diffusion of CTA membrane is only 50% higher than TFC membranes, but the calculation results in a deviation of 70% difference. FIG. 6 shows Jᵥ as a function of dilution ratio and time in FO process. Feed solution:tap water (80 L); draw solution: dialysis concentrate (0.35 L); Temperature: 25.5±2.5° C. In addition, the retention rate (RR) was defined as in Eq. (8) during the FO dilution process,

$$RR = \frac{{m}_{d , t}}{{m}_{d , 0}} \times 100 \% \qquad ( 8 )$$

wherein m_{d,0} and m_{d,t} are the initial mass of specific ion and the mass at time t of the corresponding ion, respectively. Consequently, the ion retention rates are calculated as listed in Table 5. If the retention rate is taken as the rejection of the ions in the draw solution during FO process, it is possible to compare this retention value to the rejection value in RO test.

There is clear significant improvement in the rejection in FO mode in comparison to RO mode. This phenomenon could be explained by the extra resistance incurred by the bidirectional solute diffusion across the pores in the active separation layer, thus leading to higher rejection. Although the ion retention slightly declined as the dilution ratio increased, the rejection for most of the ions were above 95%, except for K⁺. Another interesting observation is that both membranes showed very similar ion rejection in FO process for any single ion. Although the chemistry and intrinsic properties of the CTA and TFC membranes are different, retentions of divalent ions by both membranes were generally higher than those of monovalent ions, which is consistent with previous studies. These ions may be rejected by both steric hindrance and electrostatic interaction arising from their hydrated ion dimension, diffusivity (Table 6) and the negative surface charge of the membranes. Most probably, the diffusion of ions from the feed to the draw placed large enough steric resistance in the pores, resulting in generic improvement in the ion retention for both membranes.

TABLE 5
The ion retention ratios of dialysis concentrate
under different dilution ratio in FO process

| Membrane | dilution ratio | Ca²⁺ | K⁺ | Mg²⁺ | Na⁺ | Cl⁻ |
|---|---|---|---|---|---|---|
| CTA | 6 | 98.8% | 93.1% | 99.1% | 99.3% | 99.0% |
| | 18 | 95.3% | 89.2% | 97.5% | 97.2% | 97.3% |
| HF-TFC | 6 | 98.4% | 92.5% | 99.2% | 99.2% | 99.1% |
| | 18 | 94.7% | 90.8% | 97.7% | 97.6% | 98.2% |

TABLE 6
The characteristics of hydrated ions

| | Hydrated ion radius | Diffusion coefficient |
|---|---|---|
| Ion | (nm) | (10⁻⁹ m² s⁻¹) |
| K⁺ | 0.331 | 1.957 |
| Ca²⁺ | 0.412 | 0.792 |
| Na⁺ | 0.358 | 1.334 |
| Mg²⁺ | 0.428 | 0.706 |
| Cl⁻ | 0.332 | 2.032 |

### 2.4 Stability of Osmotic Dilution

Forward osmosis has been reported as being connected to slow fouling processes due to low hydraulic pressure. Based on the high flux of TFC membrane, relatively long-term process experiment (each run for about 36 h) was performed to explore the fouling behavior of the TFC membrane in FO process using tap water.

FIG. 7 shows the water flux following the dilution time of the dialysate concentrate. The cycle 1 is blank test using DI water as the feed. Afterwards, four test cycles using tap water were performed. No obvious decline in the initial FO flux was observed, only a slight and gradual decline in the end flux was found during the fourth cycle experiment. It was suspected that organic fouling was aggregated and the membrane was rinsed with NaOH at pH=10.0±0.1. Cycle 5 shows that water flux was recovered. In total, an osmotic dilution test for about 180 h was performed. The high flux recovery indicates that the new application of FO membrane for osmotic dilution of dialysate concentrate using tap water is very promising.

1-18. (canceled)

19: A hollow fiber membrane filtration device for the generation of ready-to-use dialysis fluid for use in hemodialysis by forward osmosis, the membrane filtration device comprising:
a plurality of hollow fibers axially extending through a cylindrical housing and being embedded and held, at their open ends, in a moulding compound, thereby isolating said hollow fibers from a first fluid chamber defined by the outer surface of said hollow fibers and the inner surface of said housing, wherein said first fluid chamber has a first fluid inlet and a first fluid outlet, both provided on the housing, and further comprising a second fluid inlet and a second fluid outlet in communication with a second fluid chamber defined by the interior of the hollow fibers, and
wherein the hollow fibers include a composite membrane comprising a hollow fiber support membrane including (i) 80-99% by weight of at least one hydrophobic polymer selected from the group consisting of polysulfone (PS), polyethersulfone (PES) and polyarylethersulfone (PAES), (ii) 1-20% by weight of polyvinylpyrrolidone (PVP), and (iii) a polyamide layer on the lumen side of the hollow fiber support membrane.

20: The hollow fiber membrane filtration device of claim 19, wherein the composite hollow fiber membrane is configured to allow a permeate flow rate of between 10.0 L/hm² to 20.0 L/hm².

21: The hollow fiber membrane filtration device of claim 19, wherein the composite hollow fiber membrane has a salt permeability coefficient of between 0.14 L/m² h and 0.24 L/m² h.

22: The hollow fiber membrane filtration device of claim 19, wherein the composite hollow fiber membrane has a total wall thickness of from 27 μm to 50 μm and an inner diameter of from 170 μm to 230 μm.

23: The hollow fiber membrane filtration device of claim 19, wherein the composite hollow fiber membrane comprises a hollow fiber support membrane having an asymmetric three-layer structure consisting of a dense layer on the lumen side of the hollow fiber having a thickness of below 0.6 followed by a support layer having a sponge structure and a thickness of from 1 to 15 and a third layer having a finger-structure and a thickness of from 25 to 50 and a polyamide layer having a thickness of from 5 nm to 500 nm which is present on the dense layer on the lumen side of the hollow fiber.

24: The hollow fiber membrane filtration device of claim 19, wherein the total usable membrane surface area is between 1.5 and 2.8 m² and the packing density of the hollow fibers within the housing is between 45% and 70%.

25: The hollow fiber membrane filtration device of claim 19, wherein the polyamide layer is configured to be applied to the lumen side of the hollow fiber support membrane, and consists of products by interfacial polymerisation.

26: A process for preparing the hollow fiber membrane filtration device of claim 19, the process comprising:
a) contacting the lumen side of a hollow fiber support membrane with an aqueous m-phenylenediamine (MPD) solution;
b) allowing the MPD solution to contact the surface for 1 to 4 minutes;
c) removing excess MPD solution by blow-drying the interior of the fibers with an inert gas;
d) contacting the interior side of the hollow fiber membranes with a TMC-hexane solution;
e) allowing the TMC-hexane solution to contact the surface for 0.5 to 2 minutes;
f) circulating hot water with a temperature of from 85° C. to 95° C. in the lumen of the hollow fiber membranes for 3 to 10 min to cure the nascent polyamide layer, wherein the polyamide layer consists of products of the m-phenylenediamine (MPD) and the TMC-hexane by interfacial polymerization; and
g) optionally storing the hollow fiber membranes in deionized water before further use.

27: The process of claim 26, wherein step (f) alternatively comprises storing the hollow fiber membranes or hollow fiber membrane filtration device in an oven at a temperature of 100° C.±20° C.

28: A method of preparing ready-to-use dialysis fluid from raw water by forward osmosis, the method comprising:
a) providing a hollow fiber membrane filtration device comprising a plurality of forward osmosis hollow fiber membranes axially extending through a cylindrical housing and being embedded and held, at their open ends, in a moulding compound, thereby isolating said hollow fibers from a first fluid chamber defined by the outer surface of said hollow fibers and the inner surface of said housing, wherein said first fluid chamber has an first fluid inlet and a first fluid outlet, both provided on the housing, and further comprising a second fluid inlet and a second fluid outlet in communication with a second fluid chamber defined by the interior of the hollow fibers;
b) passing raw water through the second fluid chamber of the filter device, while recirculating a liquid dialysis concentrate through the first fluid chamber in a countercurrent, thereby creating a flux of pure water from the lumen side of the composite hollow fiber membrane into the dialysis concentrate;
c) terminating recirculation upon reaching the target concentration of the dialysis solution; and
d) providing the ready-to-use dialysis fluid for use in an extracorporeal hemodialysis circuit.

29: The method of claim 28, wherein more than one hollow fiber membrane filtration devices are applied in parallel or in series.

30: The method of claim 28, wherein the liquid dialysis concentrate comprises at least one substance chosen from the group consisting of glucose, bicarbonate, potassium, acetate, lactate, citrate, pyruvate, carbonate, magnesium, calcium, sodium, sulphate, phosphate, chloride and amino acids.

31: The method of claim 30, wherein the liquid dialysis concentrate comprises at least one substance chosen from the group consisting of acetate, lactate, citrate and glucose.

32: The method of claim 30, wherein the liquid dialysis concentrate comprises at least one substance chosen from the group consisting of bicarbonate, sodium and chloride.

33: The method of claim 28, wherein at least one electrolyte detector is provided in the circuit, optionally after the hollow fiber membrane filtration device.

34: A system for preparing ready-to-use dialysis fluid, the system comprising:
(a) a hollow fiber membrane filtration device comprising a plurality of forward osmosis hollow fiber membranes axially extending through a cylindrical housing and being embedded and held, at their open ends, in a moulding compound, thereby isolating said hollow fibers from a first fluid chamber defined by the outer surface of said hollow fibers and the inner surface of said housing, wherein said first fluid chamber has an first fluid inlet and a first fluid outlet, both provided on the housing, and further comprising a second fluid inlet and a second fluid outlet in communication with a second fluid chamber defined by the interior of the hollow fibers;
(b) a dialysis concentrate feed line connected to the inlet of the first fluid chamber of the hollow fiber membrane filtration device for providing dialysis concentrate from a container;
(c) a dialysis concentrate return line connected to the outlet of the first fluid chamber of the hollow fiber filtration device for returning the diluted dialysis concentrate to the container;
(d) a raw water supply line connected to the inlet of the second fluid chamber of the hollow fiber membrane filtration device;
(e) a raw water effluent line connected to the outlet of the second fluid chamber of the hollow fiber membrane filtration device; and
(f) an electrolyte detector in the dialysis concentrate return line,
wherein the hollow fiber members consist of a composite membrane comprising a hollow fiber support membrane including (i) 80-99% by weight of at least one hydrophobic polymer selected from the group consisting of polysulfone (PS), polyethersulfone (PES) and polyarylethersulfone (PAES), (ii) 1-20% by weight of polyvinylpyrrolidone (PVP), and (iii) a polyamide layer on a lumen side of the hollow fiber support membrane.

35: The system of claim 34, further comprising a control system configured to run the preparation of the ready-to-use dialysis fluid, comprising pumping means for passing raw water through the second fluid chamber of the hollow fiber membrane filtration device and for recirculating dialysis concentrate through the first fluid chamber of the hollow fiber membrane filtration device, the control unit being connected to at least one electrolyte detector and configured to receive a value representative of the conductivity of the dialysis fluid, wherein said control unit is configured to terminate the recirculation of the dialysis concentrate at a set conductivity value which indicates that the target concentration of the ready-to-use dialysis fluid has been reached.

36: The system of claim 34, wherein the conductivity of the dialysis fluid or the concentration of at least one substance in the dialysis fluid is determined, said substance including sodium.
